(19) United States
(12) Patent Application Publication
(10) Pub. No.: US 2022/0096859 A1
(43) Pub. Date: Mar. 31, 2022

(54) Portable Stimulation Systems and Methods

(71) Applicant: Wave Neuroscience, Inc., Newport Beach, CA (US)

(72) Inventors: James William Phillips, Fountain Valley, CA (US); Alexander J. Ring, Newport Beach, CA (US); Erik Won, Fullerton, CA (US); Alfred Jennings Walke, Encinitas, CA (US); Spencer Vigoren, Newport Beach, CA (US); Edward James Mason, San Diego, CA (US)

(21) Appl. No.: 17/449,661

(22) Filed: Sep. 30, 2021

Related U.S. Application Data
(60) Provisional application No. 63/085,562, filed on Sep. 30, 2020.

(51) Int. Cl. A61N 2/06; A61N 2/00

(52) U.S. Cl. CPC A61N 2/06; A61N 2/004

(57) ABSTRACT

Exemplary embodiments described herein include a method of administering a simulation energy to the user. The stimulation energy may be any combination of electrical, magnetic, light, sound, or vibrational energy. The stimulation energy may be applied at a frequency. Exemplary embodiments may include any combination of interfaces, instructions, or controls for controlling the stimulation energy and/or providing information about the system described herein. For example, a mobile device may be used as a handheld controller that may communicate wireless to a head mountable device for administering stimulation energy. Exemplary embodiments of the head mountable device may also include electrodes for detective electrical activity of a user.

### PRIORITY

The instant application claims priority to U.S. Provisional Patent Application No. 63/085,562, filed Sep. 30, 2021, which is incorporated by reference in its entirety herein.

### BACKGROUND

Repetitive Transcranial Magnetic Stimulation (rTMS) and transcranial Alternating Current Stimulation (tACS) have been used to improve symptoms of mental disorders and to modify brain function. rTMS uses high energy magnetic pulses from a magnetic field generator that is positioned close to a person's head, so that the magnetic pulses affect a desired treatment region within the brain. tACS uses electric current pulses delivered to the scalp. Traditionally, the rTMS or tACS pulses are generated at a fixed frequency for a short time duration. For example, a typical rTMS system may generate pulses at 10 Hz for a duration of 5 seconds. A series of pulses generated over a period of time is referred to as a pulse train. An rTMS treatment session may be composed of several pulse trains, with a rest period between each pulse train. A typical rest period may be 55 seconds, such that 5 seconds of rTMS pulses are generated per minute.

Examples of energy stimulation may be found in, for example, U.S. Pat. Nos. 8,456,408; 8,475,354; 8,480,554; 8,585,568; 8,870,737; 8,926,490; 9,015,057; 9,308,385; 9,649,502; 9,962,555; 10,342,986; 10,350,427; 10,398,906; 10,420,482; and 10,420,953; and US Publication Nos. 2016/0045756; 2017/0296837; 2018/0104504; and 2018/0229049, each of which is incorporated by reference in their entirety herein.

### DRAWINGS

FIG. 1 illustrates an exemplary system according to embodiments of the invention in use by a user.

FIGS. 2-4 illustrate exemplary perspective views of a head mounted device (HMD) according to embodiments described herein.

FIG. 5 illustrates an exemplary head mount device with the exterior portion removed to permit viewing of the interior components.

FIG. 6 illustrates an exemplary system according to embodiments described herein.

FIGS. 7A-7C illustrate an exemplary user and representative positioning according to embodiments described herein.

FIG. 8 illustrates an exemplary component diagram of a portion of an HMD according to embodiments described herein.

FIG. 9 illustrates an exemplary user and representative positioning according to embodiments described herein.

FIG. 10 illustrates an exemplary system according to embodiments described herein.

FIG. 11 illustrates an exemplary waveform and wavelet according to embodiments described herein.

### DESCRIPTION

The following detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention. It should be understood that the drawings are diagrammatic and schematic representations of exemplary embodiments of the invention, and are not limiting of the present invention nor are they necessarily drawn to scale.

Exemplary embodiments described herein include a head mounted device for providing stimulation to a user. The stimulation device may be of one or more energy forms. For example, a stimulation device may be a magnetic energy, electric energy, vibrational energy, light energy, ultrasound, radio frequency, acoustic, and combinations thereof. In an exemplary embodiments, the magnetic energy is a magnetic field having a frequency. In an exemplary embodiment, the electric energy is an electrical current having a frequency. The frequency of the energy source may be constant, variable, random, constant for a duration, and combinations thereof. Exemplary embodiments include systems configured to and methods administering energy as described herein at a specific frequency or combination of frequencies in order to influence an intrinsic frequency of the EEG band of the user. In an exemplary embodiment, the system and methods include selecting an energy source to pull the intrinsic frequency of the user to a desired band.

Exemplary embodiments described herein may include a portal device. The head mount device and system thereto may include a head mounted device, a controller, and/or a user interface. The system may be of a shape and weight configured to be portable and easily moved for use in different locations. The system may be configured for use at home by a user. The system may be configured to be used by a user without assistance of another person.

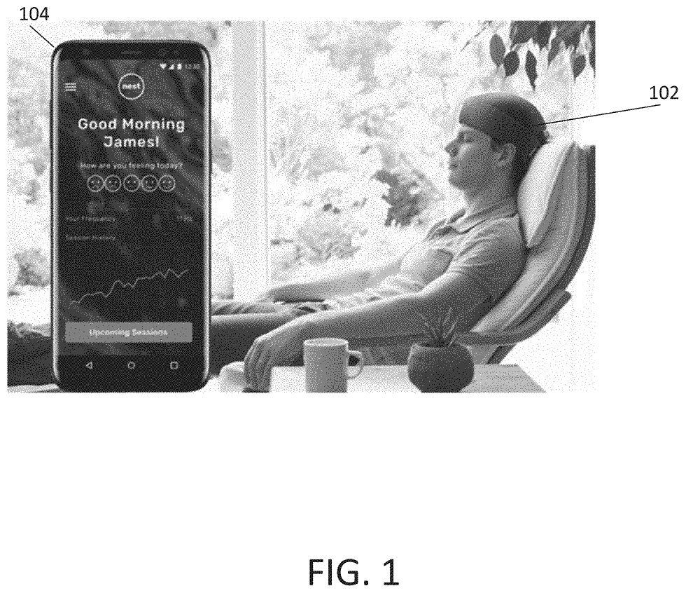

FIG. 1 illustrates an exemplary headset system for stimulation activities according to embodiments described herein. Exemplary embodiments of a system described herein includes a head mounted device (HMD) 102 to be worn by a user. Exemplary embodiments of a system described herein includes a controller 104 that permits the use of the system and/or the application of stimulation through the HMD.

Exemplary embodiments described herein include a method of administering a simulation energy to the user. The stimulation energy may be any combination of electrical, magnetic, light, sound, or vibrational energy. Exemplary embodiments described herein include magnet stimulation using three rotating diametrically magnetized cylindrical magnets to generate an alternating magnetic field near the user's head. The magnetic field frequency may be set or controlled based on any combination of interfaces, instructions, or controls described herein. For example, a mobile device may be used as a handheld controller 104 that may communicate wireless to the HMD 102. The HMD and/or controller may communicate directly with each other, and/or may communicate through a local and/or remote communication device. An exemplary communication between the handheld controller and the headset may be wired and/or wireless, such as through Bluetooth. For example, the handheld controller 104 may communicate over a wifi or cellular network to communicate with a remote server, while the HMD similar communicates over wifi or other network interface to similarly communicate with the remote server. The remote server may therefore act as an intermediary between the handheld controller and the application of a stimulation energy through the HMD.

In an exemplary embodiment, the method includes conducting a session of applying stimulation to a user through the HMD. The session may last for a defined period of time. The user may be informed by the any combination of the controller, HMD, indicator, or other interface any combination of the amount of time remaining, the end of a session, time until an end of a session, approximate period to an end of a session, the present status of the session, or other information about the session. For example, a timer, color code, light intensity, light or timer bar, gauge, or other visual indicator may be used to indicate whether a user is at the beginning of session, middle of the session, or toward an end of a session. Other indicators may be configured to indicate whether a user is approaching an end of a session, whether the session is paused, whether the stimulation is being applied, whether the stimulation is not being applied, etc.

Exemplary embodiments described herein may include a recording system. The recording system may retain information about the user and/or sessions. For example, a duration, period, frequency, energy strength, energy source, EEG signal, and combinations thereof may be recorded for each session. Exemplary embodiments may include a processor for analysing, recording, retrieving, and/or presenting the information to a user. For example, the system may be configured to generate a usage report for the user. Other reports may also be provided, such as an evolution or progression of the user's EEG, or an attribute from the EEG.

In an exemplary embodiment, a head mount device may include an inner portion 402 and an outer portion 404. In an exemplary embodiment, the inner portion and outer portion are configured to fit together. The inner portion may be detachable or coupled to the outer portion. The attachment may be removable. In an exemplary embodiment, the inner portion may be configured to house one or more of the energy sources described herein. In an exemplary embodiment, the outer portion may be configured to secure the inner portion in place, provide cover and comfort to the user, provide aesthetic appeal, and combinations thereof. In an exemplary embodiment, different outer portions may be interchangeable to permit customization, fit, color, form, and combinations thereof. For example, different hat configurations may be used as an outer portion. In an exemplary embodiment, materials of the outer portion may be selected for fit, attachment, and/or comfort.

Even though the system is shown and described as having a head mounted device, the system is not so limited. Exemplary embodiments may incorporate energy stimulation devices according to embodiments described herein into other objects. For example, energy stimulation devices may be incorporated into a headrest of a car or a plane. Exemplary embodiments may incorporate stimulation device described herein into a recliner or chair. Exemplary embodiments may incorporate stimulation devices into a pillow. Other devices that are positioned near the head of a user may also be used and taken advantage of to incorporate energy stimulation devices according to embodiments described herein.

In an exemplary embodiment, the HMD 200 may include a feature to control and/or adjust a size of the HMD. In an exemplary embodiment, the HMD is adjustable to accommodate head sizes of approximately 21 inch circumference to approximately 25 in circumference. In an exemplary embodiment, the HMD may approximate hat sizes of approximately 6¾ to 7¾.

In an exemplary embodiment, the HMD 200 may include a feature to secure the HMD to a user's head. Preferably, the HMD is configured to be positioned and secured to the user's head. During movement of the user, such as in tilting their head, fully or partially forward, backward, and/or side to side, the HMD may remain on the user's head. In an exemplary embodiment, the feature to secure the HMD retains the HMD in a position relative to the head such that the magnets remain in approximately the same position. In an exemplary embodiment, the maintenance of the relative position of the HMD to a user's head is preferably a movement relative to the head of equal to or less than ¼ of an inch when a user moves their head, and/or tilts/rotates their head by up to 20 degrees to the front, back, or either side.

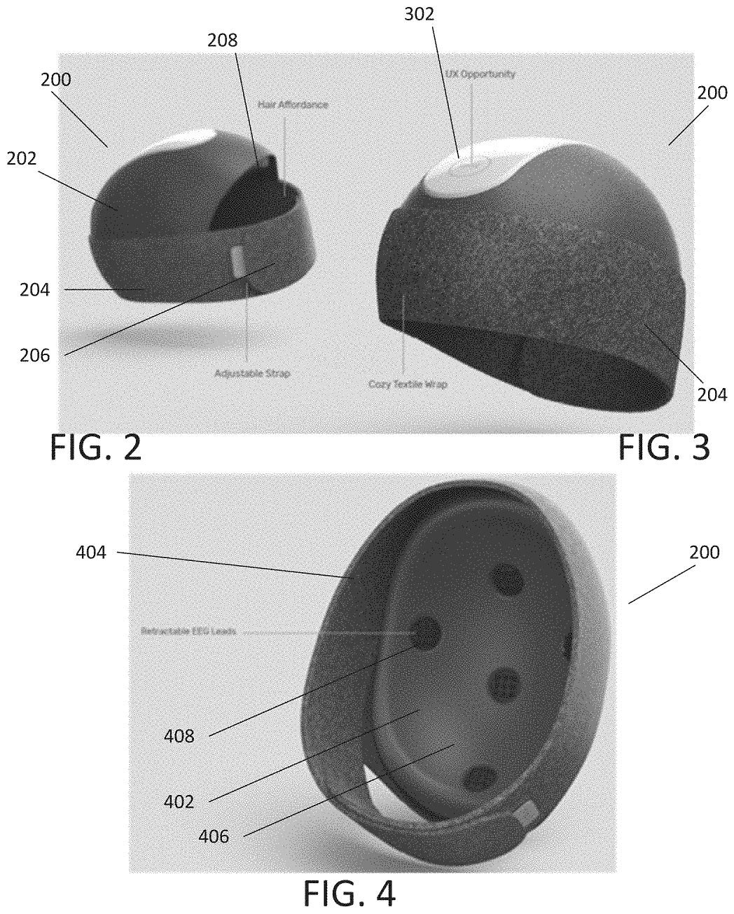

As illustrated in FIGS. 2-4, exemplary embodiments of the HMD may include a top portion 202 and a band 204 circumscribing the lower portion of the HMD. The top portion 202 may cover a majority of the top of the head above the band. The band 204 may be around a lower terminal end of the HMD. The band may provide a flexible contour to comfortably conform to the user's head. The band 204 may comprise a flexible material. The band 204 comprise a fabric, woven, non-woven, flexible material. The band 204 is configured to circumscribe the user's head. The band may facilitate the adjustable fit, the retention to a user's head, and/or securement against relative movement during user movement. Although described as a band, a band is not intended to be limiting to a specific shape configuration. The band may be a generally elongated strip that is positioned about a lower portion of the HMD. The band may also fully and/or partially cover the head of the user.

In an exemplary embodiment, the band 204 may comprise a portion that defines an inner diameter of the HMD. The band 204 may accommodate variable dimensions. For example, the band 204 may be elastic and/or stretchable in one or more directions. The band may therefore permit and accommodate various head sizes and/or shapes by deforming, flexing, and/or stretching. As illustrated, the band 204 may include an adjustment feature 206 to permit a variable internal diameter. As illustrated in FIG. 2, the adjustment feature 206 is an overlap of the strap onto itself and an attachment therebetween. The attachment may be through a hook and loop fastener (such as Velcro®), snaps, projections mated with apertures, hook and eye, or other fastening devices as known to a person of skill in the art.

In an exemplary embodiment, the HMD may be configured with an opening 208. The opening 208 may work in conjunction with the adjustment of the strap 204 to create different inner dimensions of the system. In an exemplary embodiment, the opening 208 may be used as an opening for hair. The opening 208 may be between the top portion 202 and the band 204. In an exemplary embodiment, in order to provide a close fit to the head by the inner portion 402, the hair of the user may be directed out one or more apertures of the HMD.

In an exemplary embodiment, the HMD may have a user interface. In an exemplary embodiment, a user interface may include input controls, output indicators, output devices, and combinations thereof. For example, the HMD may include buttons, knobs, sliders, touch interfaces, smart buttons, and combinations thereof. The HMD may include lights, sounds, and combinations thereof. As illustrated in FIG. 3, for example, the HMD may include a user interface 302. In an exemplary embodiment, the UX 302 may be an output indicator 302. The output indicator may include a light source for providing light in different colors to a user. In an exemplary embodiment, the UX 302 is a smart touch interface. The smart touch interface may include proximity and/or pressure sensors to determine a touch and/or motion. The touch and/or motion may control one or more features described herein. For example, a touch (like a press of a button) may be used to turn the HMD on and off. A touch that is in the form of a swipe in a circle (such as in turning a wheel) may increase or decrease a feature described herein (such as an amplitude of an energy source and/or frequency of an energy source and/or mode of an energy source. In an exemplary embodiment, a swipe and/or combination of touches may change the selection of an energy source. Other combinations of touches and/or motion may also be used to control a feature of the HMD. For example short touches may have one function, while press and hold or longer touches may have another functional control or effect.

As illustrated in FIG. 4, the inner portion 402 is configured to retain, house, and/or position the energy sources as described herein. The inner portion 402 may have a rigid and/or semi-rigid form such that the relative positions of the energy sources may be generally known and maintained. The inner portion 402 may be configured to support the energy sources and provide sufficient infrastructure to retain the energy sources in approximately the same location during a treatment session.

In an exemplary embodiment, the inner portion 402 may include indentations and/or apertures 404. These indentations may be used to position an interface for one or more of the energy sources. For example, the indentations may permit the attachment of an electrode, magnetic source, vibrational source, light source, and combinations thereof. In an exemplary embodiment, a plurality of indentations are provided to permit the attachment of different energy sources in different combinations within the HMD. Although described herein as providing support and/or attachment of an energy source, this is also intended to include an energy receiver. For example, an electrode may be used to stimulate a user by providing an electrical current to the user or an electrode may be used to receive an electrical signal from the user and detect activity of the brain from the user. Both electrode configurations of an energy transmission device and/or an energy receiving device are within the understanding and scope of an energy source. As illustrated, five indentations are suggested with three positioned along the central axis of symmetry from front to back of the HMD, and one more on each side of the center axis. However, any combination of indentations may be used to provide additional combinations of energy source locations.

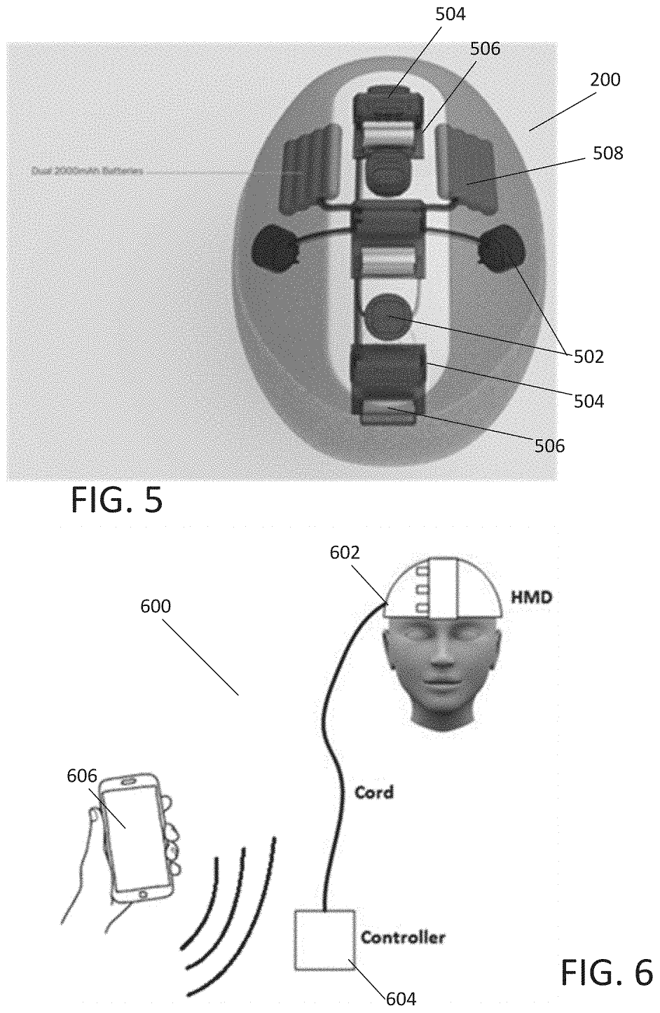

FIG. 5 illustrates an exemplary interior view of a HMD according to embodiments described herein. In an exemplary embodiment, the system may include a power source 508, motors and/or controllers 504, and one or more energy sources 506, 502. As illustrated, the system may be a combination of energy sources. For example, the system may include a magnetic energy source 506 and an electrical energy source 502. The magnetic energy source may be a permanent magnet 506 coupled to a motor 504 for rotating the magnet about an axis. The electrical energy source 502 may be an electrode. Batteries 508 may be used to power the system.

In an exemplary embodiment, the HMD may comprise different fabrics and/or materials. For example, materials may be incorporated into the HMD to enhance magnetic stimulation. As an example, materials may be incorporated into the HMD to permit electrical conductivity. The materials may be incorporated to provide leads from one or more of the apertures. The materials may be selected or may incorporate properties to enhance hygiene. For example, surface textures may be provided, materials may be selected, coatings may be provided, or other incorporation may be made to improve the microbial resistance of the system described herein, and/or to permit easier cleaning.

Exemplary embodiments, may include one or more component parts to be modular. The modular component part may be removable from the rest of the system. The modular component part may be replaceable for easy maintenance. The modular component part may be disposable to promote interchangeable use between different users. The modular components may permit customization between users, such as for size, appearance, and feature accommodations. For example, different modular components may include different features (such as for different modes of stimulation—light, audial, visual, magnetic, electric, vibrational, etc.; different user interface features; different sizes; different aesthetic features; different materials to provide temperature control such as for warm or cool environments).

Materials or components may be used to provide rechargeable power. For example, solar panels and/or flexible solar material may be incorporated into any of the system components described herein.

In an exemplary embodiment, the head mounted device (HMD) 200 may include a housing 402. The housing may enclose the electronics, controllers, motors, magnetic energy sources, electric energy sources, and/or vibrational energy sources, and combinations thereof. The housing may enclose the components so that a user cannot access the internal components. The internal components may be protected from humidity, sweat, dirt, hair, etc. The housing may also protect the user from the internal components. The housing may also support and/or permit some components to be exposed. For example, electrodes may be supported on an outer surface of the housing to provide contact with a user.

As described above, the housing may include apertures and/or indentations for use to support removable electrodes. The electrodes may be friction fit into the apertures, screwed into the aperture, retained through magnetic attraction, or other form of retention feature. As illustrated in FIG. 4, an exemplary embodiment permits five electrodes to be supported on the HMD to provide an assessment of the electrical activity of a user's brain. The electrodes may be positioned to receive electrical signals from the brain for determining an intrinsic alpha frequency of the user.

In an exemplary embodiment, the electrodes may be retractable. In this case, a control mechanism may be used to radially translate the electrical toward and/or away of the user's head. In this case, the electrodes may be in selective contact with the user's head. The retraction/extension of the electrode may permit greater contact and use with various user's head shapes. The retraction/extension of the electrodes may permit selective use of the electrodes and remove contact with the user when not in use.

Exemplary embodiments of the system described herein may permit a feedback loop for controlling the administration of the energy sources. For example, the electrodes may be used to receive electrical signals from the user. The received electrical signals may be used to determine an attribute of the electrical signals. In an exemplary embodiment, the retrieved signal is analysed to determine an alpha wave frequency of the user. The received electrical signal of the user may then be used to set a parameter of the next treatment session. For example, based on the analysed alpha wave frequency of the user, the system may be configured to select a combination of the energy sources to use on a patient and/or in an attribute of the energy source (such as its frequency and/or amplitude). In an exemplary embodiment, the electrodes may be used to retrieve electrical signals from the user before treatment, during treatment, within intervals of treatment, after treatment, and any combination thereof. The retrieved electrical signals may be analysed to determine an attribute of the next treatment session, alter treatment during a session, or provide feedback to the user about a condition of the user.

As illustrated in FIG. 1 the system may include a user interface 104 for receiving information about a treatment session and/or for controlling a treatment session. In an exemplary embodiment, the user interface may comprise an application downloaded on a mobile device and when executed by a processor of the mobile device is configured to perform the functions described herein. Although the user interface is shown and described as an application run on a user's mobile device, embodiments of the description are not so limited. Instead, other user interfaces may also be used, such as a remote control, buttons, toggles, etc.

Exemplary embodiments described herein may include any combination of controllers as described herein. For example, any of the user interface, controller, or HMD may include mechanical and/or electrical control interfaces. For example, buttons, sliders, toggles, switches, rotary wheels, knobs, and other user input devices may be used to control a mode of operation, an amplitude, a frequency, a duration, and any combination thereof. Other user input and output interfaces may also be used. For example, exemplary embodiments may include voice controls.

Exemplary embodiments described herein may include options to provide a status to a user on the status of a session. For example, light combinations on any of the HMD, controller, user interface, or other system component may be used to indicate a time within a treatment session. Various indicators may be used including colors, haptic responses, sounds, icons, colors, and/or other visual or sensory indicators.

Exemplary embodiments described herein may include other sensory enhancements. For example, the system may include visual cues to improve relaxation. The system may include light projections that may be used to project light and/or shapes onto the environment around the user. Other sensory enhancements may include audial enhancements. For example, the system may include soothing sounds, music, white noise, etc.

Exemplary embodiments of the user interface described herein may permit user feedback. The user feedback may be used to set a parameter of the treatment session. For example, a user may provide feedback on the effectiveness of a treatment, or in their general mood or condition being treated. The system may, for example, ask a user to enter an anxiety level or general mood before, during and/or after a treatment session. The system may use the user feedback to adjust a treatment protocol.

Exemplary embodiments analyse the information received and/or provided by the system. The system may be configured to provide reports on treatment sessions, feedback conditions, user received signals (such as those received from the electrodes to detect electrical activity of the user).

Exemplary embodiments described herein permit the user interface to control the treatment session. In an exemplary embodiment, the user interface may permit a user to purchase treatment sessions. For example, the user may engage with the user interface and indicate a number of treatment sessions, and total amount of treatment, and/or a desired target to reach a given condition. The system may thereafter be configured to purchase treatment session time corresponding to individual use sessions or in block sessions. The system may therefore determine whether a treatment session has been paid for before administering a session. If the session has not yet been paid for, the system may request payment before proceeding. The system may interact with third party payment systems to permit payment from the user to the treatment protocol system. The system may also interact with other medical information and/or providers. For example, the system may interact with insurance exchanges in order to initiate an insurance payment request for a treatment session.

FIG. 6 illustrates an exemplary system according to embodiments described herein. As illustrated the energy stimulation system 600 includes a head mounted device (HMD) 602. The HMD 602 may be controlled by a controller 604. The controller 604 may use any combination of interfaces. For example, the controller 604 may have a user input/output or user interface directly integrated into the housing of the controller 604. For another example, the controller 604 may communicate either wirelessly or wired to a user input/output or user interface. As illustrated, the controller 604 communicates to a user handheld interface (smart phone) 606 wirelessly through, for example, Bluetooth.

Although the exemplary embodiment of FIG. 6 separates the head mounted device from the controller, the system is not so limited. Exemplary embodiments include the controller (and/or components thereof such as the battery) incorporated into the head mounted device.

Although the exemplary embodiment of FIG. 6 separates the controller from the user interface, the system is not so limited. Exemplary embodiments include the controller (and/or components thereof) incorporated with the user interface. For example, a touch screen or other user input/output devices, such as buttons, knobs, sliders, displays, lights, and any combination thereof may be incorporated into the controller.

Exemplary embodiments may remove some of the electronic and/or mechanical components from the HMD in order to reduce weight, and limit the size of the device positioned and/or supported on the user's head. For example, the batteries or power may be provided within a controller housing separate from the headset housing. Other components may include the communication interface to communicate with one or more other electronic devices (such as the mobile device and/or remote server) as described herein. Other components may include memory and/or processor for controlling portions of the system described herein. Exemplary embodiments may include software to control major functions of the device, such as turning motors on and off, setting frequencies, recording session information, retrieving signals from a user, and combinations thereof. The system may therefore include memory for storing the software instructions in a non-transient machine readable format that, when executed by a processor, perform the functions described herein. The memor(y/ies) and/or processor(s) may be in the controller housing 604, at the HMD 6-2, handheld device 606, remote server (not shown), and combinations thereof. In an exemplary embodiment, the HMD support and worn by the user on a user's head is less than or equal to approximately 3.5 pounds.

The controller 604 may include any combination of system mechanical and/or electric components to operate the HMD 602. The controller 604 may be coupled to the HMD 602 through a cord, for example. As illustrated, the cord may be sufficiently long that the HMD can be worn by a user, and the controller positioned on a secure surface, such as a table, floor, arm chair, user's lap, etc. In an exemplary embodiment, the controller 604 and/or HMD 602 may include a cord management system. One or both of the controller 604 or HMD 602 may include an interface to recoil, wind, or otherwise retain a portion of the cord that is not necessary for the positioning of the controller away from the HMD. In an exemplary embodiment, the system may include a cord lock, extension, and/or retraction system to automatically retract and/or retain the cord at a desired length.

In an exemplary embodiment, the controller 604 may be easily transportable. The controller may therefore weight less than or equal to 3.5 pounds. In an exemplary embodiment, the controller 604 is less than or equal to 8 inches cubed. In an exemplary embodiment, the controller 604 is equal to or within dimensions of approximately 2 inches by 3 inches by 8 inches.

In an exemplary embodiment, the controller 604 is coupled to the headset by a cable. The cable may provide power and/or data transmission between the HMD 602 and the controller 604. The cable may be at least 36 inches long. The cable may be less than approximately 0.5 pounds. In an exemplary embodiment, the cable is detachable from either or both of the HMD 602 and/or the controller 604.

In an exemplary embodiment, the controller may provide power to the HMD. In an exemplary embodiment, the controller may include rechargeable batteries. The controller may therefore also include a power supply cord. The controller may be plugged in to receive power. The controller may then provide power to the HMD. In an exemplary embodiment, the power to the controller may recharge the batteries. The controller may power the electronics of the controller and/or HMD from the batteries. In an exemplary embodiment, the power to the controller and the HMD from the batteries may not operate unless the controller is unplugged from AC power.

In an exemplary embodiment, the controller 604 may communicate with a user interface 606. In an exemplary embodiment, as described herein, the user interface is a handheld mobile device, such as a smart phone, electronic pad, tablet, or smart device. The user interface may also be provided on other electronic devices such as a touchscreen, smart tv, electronic pad, smart device, or other user interface having input and/or output features. In an exemplary embodiment, the controller 604 may communicate by passing information to and from the user interface 606.

In an exemplary embodiment, the system may be configured to have an indicator to the user. The indicator may be on the controller 604. The indicator may be, for example, on or more lights, digital display, screen, or other indicator. The controller may have one or more indicators. The indicators may identify whether the HMD is turned on or off, whether HMD is emitting a source of energy, what form of energy the HMD is admitting (electric, magnetic, vibrational, etc.), what the status of the wireless link (pairing, unpaired, paired, etc.), status of the energy administration session (ready to start, starting, during session, nearing end of session, end of session, etc.), duration or time of a session (elapse time, remaining time, total session time, total use time, etc.), and any combination thereof.

In an exemplary embodiment, the controller may include a microprocessor based system. The controller may include a clock-module for providing timing information to the microprocessor. The controller may have internal memory. The controller may include a port for external memory. For example, the controller may access flash memory removably coupled to the controller.

In an exemplary embodiment, the controller may be configured to control a session. For example, the controller may be configured to start, stop, pause, set a frequency, and combinations thereof. In an exemplary embodiment, a treatment session comprises a specified duration. The controller may therefore automatically terminate or end a session at the end of the specified duration without user intervention or further user input after the session is started.

In an exemplary embodiment, the system, including any combination of the HMD 602, controller 604, and user interface 606 is configured to be operated by an individual without the assistance of another person.

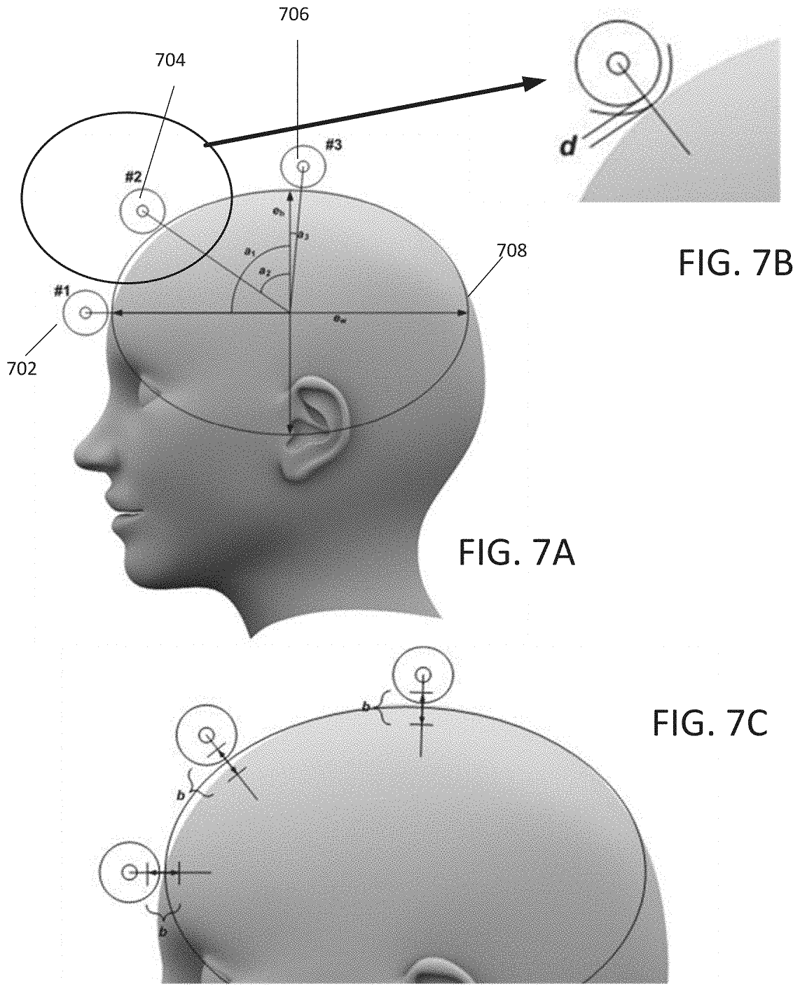

FIGS. 7A-7C illustrate exemplary positioning of stimulation energy generation devices according embodiments described herein. In an exemplary embodiment, the HMD may include any combinations of stimulation energy generation devices.

In an exemplary embodiment, a stimulation energy may be administered at a target frequency. The stimulation energy may be a sinusoidal waveform. In an exemplary embodiment, the administered stimulation energy may be applied at a constant frequency. The administered stimulation energy may be at different frequencies. For example, the system may administer a stimulation frequency for a first duration at a measured frequency (or a harmonic or subharmonic thereto), and a second duration at a target frequency toward a desired frequency to be achieved by the user (or a harmonic or subharmonic thereto). Other combinations of frequencies may also be used. For example, a measured frequency of a user may be obtained. The system may be programmed with and/or determine a target frequency in which the user is attempting to obtain (such as an average frequency or identified normal frequency of a user group). The administered frequency may therefore include an offset from the measured frequency in a direction toward the target frequency, such that the user is administered a stimulation energy between the measured frequency and the target frequency. The procedure may include changing the frequency from the first administered frequency to a second administered frequency where the second administered frequency is between the first administered frequency and/or equal to the target frequency. Therefore, the procedure may use sequential administration frequencies that incrementally moved from the measured frequency to a target frequency. Other combinations of frequencies may be used. In the event of one or more stimulation sources are used (whether of the same energy type or not), the system may administer different frequencies simultaneously and/or sequentially. The system may also administer a stimulation frequency, such as by randomly hopping a frequency about a target frequency.

Exemplary embodiments may include providing a plurality of the same and/or different energy sources. The system and method may selective administer energy from a subset of the plurality of energy sources. The subset may depend on the desire type of energy to administer. The subset may depend on the location of the energy source and a target location within the brain to be stimulated. Exemplary embodiments may selectively activate a subset of energy sources in order to target regions of the brain that are showing the least rhythmic activity, and are therefore in the most need of stimulation. In an exemplary embodiment, different subsets of energy sources may be administered at different frequencies. The different frequencies may be complementary and/or interfering. In an exemplary embodiment, a first subset of permanent magnets may be rotated at, for example 2 HZ, with another permanent magnet rotated at 4 Hz. Another embodiment may have a first magnet rotated at 8 HZ and another magnet rotated at 1 kHz.

In an exemplary embodiment, the stimulation energy generation devices may be fluctuating magnetic field generation devices. The fluctuating magnetic field may be generated through one or more rotating and/or translating permanent magnetic fields. The fluctuating magnetic field may be generated through one or more electrical coils. The permanent magnet may be rotated continuously at an administration frequency. The permanent magnet may be rotated at the administration frequency for a duration. In an exemplary embodiment, the administered stimulation energy may be administered at different duty cycle. For example, instead of a continuous rotation, for the embodiment of a permanent magnet, the permanent magnet may spin very quickly for a single rotation and remain still for the remainder of the stimulation period. More specifically, as an example only, the stimulation frequency may be 10 Hz (100 milisecond (ms) period). The magnet could rotate extremely quickly and finish a full rotation in 1 msec. The magnet may thereafter remain unrotated or stopped the remaining 99 ms before it spins gain. In this way, the rotating magnet may replicate or approximate a pulsed magnetic field generated by a standard rTMS system. Other rotational configurations may also be used. For example, a magnet could continuously spin. The magnet may spin at a desired or set frequency for a specific duration, and at a second or sweeping frequency for a second duration. In an exemplary embodiment, if the energy stimulation frequency is at 10 Hz, the magnet may spin at 1 kHz. The magnet may then in a desired duration, such as every 100 ms spin at a rate of greater than 1 kHz for a short duration, such as from 1 to 5 ms. The magnet may therefore provide a stimulation energy at a desired frequency with another frequency signal to match the peak of a slower frequency.

In an exemplary embodiment, the HMD may include three (3) permanent magnets to generate an alternating magnetic field. The magnets may be magnetized neodymium cylindrical magnets rotated to generate a magnetic field having a frequency, such as between 8.0 Hz to 13.0 Hz. As represented in FIG. 7A, the interior surface of the HMD may approximate a portion of an ovoid. As seen in FIG. 7C, the one or more magnets may be positioned such that a portion of the magnet is at, on, or approximate to the ovoid. In an exemplary embodiment, the ovoid may be an ellipse whose width is between approximately 5.2 and 5.3 inches and height between approximately 6 to 10 inches. In an exemplary embodiment, the width may be approximately 5.5 inches and the height approximately 8 inches. As illustrated, the surface of one or more magnets is approximately positioned on the ovoid surface.

In an exemplary embodiment, the magnetic stimulation may be supplied from positions on approximately a quarter of the ovoid. The magnetic stimulation may therefore, be generally applied to the forward portion of a user's head. As illustrated in FIG. 7A, a first magnet 702 may be positioned at the front of the user's head. The position may be at or proximate to an apex of the ovoid. The position of the first magnetic energy stimulation device may be over a forehead of a user. A magnet 706 may also or alternatively be positioned at the top of a user's head. The position may be at or proximate to an apex of the ovoid. The apex of the ovoid may be at the minor axis of the ovoid. The magnet 704 may be positioned alone or in combination with any of the magnet 702 and/or magnet 706. The magnet 704 may be positioned between the forward position of magnet 702 and the top position of magnet 706. In an exemplary embodiment, the magnet 704 is closer to the forward magnet 702 than the top magnet 706. In an exemplary embodiment, if the magnets are positioned along or adjacent to the ovoid, and a rotational measurement is taken from the axis out of the top of the user's head, the forward magnet 702 may be approximately positioned at 88 to 92 degrees, the middle magnet 704 may be positioned at approximately 52-56 degrees, and the top magnet may be positioned at −2.5 to −5 degrees.

Exemplary embodiments of the stimulation devices may be adjustable in their position. For example, the magnetic energy devices of FIG. 7C may be adjusted in a radial direction about the surface of the ovoid. As illustrated, a surface of the magnet may be radially positioned above or below the ovoid surface by 0 to 1 inches above or below the ovoid surface, toward or away from the user's head. To control the size of the HMD, different adjustment lengths may be permissible, such as by approximately 0.5 inches, or 0.2 inches. The adjustment may be achieved using a spring on a sliding mechanism and a hand-tightened locking screw. When the screw is loosened, the spring pushes the magnet to its most distant position on the sliding mechanism. The screw may be unlocked before the HMD is placed on the user's head, allowing the magnet to move radially on the sliding mechanism to the end position. When the user places the HMD on their head, the magnet may shift to adjust so that the magnet is as close as possible to the person's head, while still fitting comfortably. The locking screw may then be tightened, locking it in place. Alternately, the locking screw may not be tightened, or may not exist, allowing the magnet to adjust to minor shifts of the HMD on the user's head.

Other examples exist to adjust the magnet, including an adjustment screw, in which the user may wear the HMD, and adjust the screw until the magnet is in the closest position possible to the user's scalp. In another example, the magnet is on a sliding mechanism that does not comprise a spring. The user may wear the HMD and the magnet position is adjusted manually by sliding it up or down to the correct position and locking the magnet into place.

In an exemplary embodiment, the ovoid configuration defines an interior surface of the HMD. In an exemplary embodiment, the interior surface of the HMD is intended to contact a user's head. Therefore, one or more of the energy sources may be removed away from the user's head from the interior surface of the HMD. As illustrated in FIG. 7B, a distance between the active surface configured to contact a user's head and the surface of the magnet may be less than or equal to approximately ¼ inch. In one embodiment, the distance, d, is less than or equal to ⅛ of an inch.

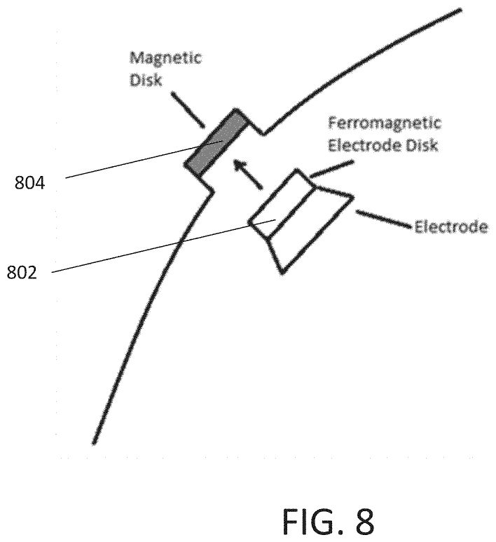

In an exemplary embodiment, the head mounted device (HMD) may include electrodes for recording electrical signals of the user's body/brain. For example, three electrical leads may be used to couple to a user's head through the HMD. As illustrated in FIG. 8, the electrodes may be detachable from the HMD. In an exemplary embodiment, the electrode 802 may include a magnetic backing. The HMD may include one or more indentations and/or apertures for supporting and positioning one or more electrodes. The aperture and/or indentation for the electrode may include a magnetic component. The electrode, with a magnetic component may be attracted to the headset and remain in place. In an exemplary embodiment, the electrode may be removable, replaceable, and/or disposable. In an exemplary embodiment, the electrode may make an electrical connection with an EEG amplifier when positioned in the HMD and attached to the magnet in the HMD.

Exemplary embodiments described herein may detect an electrical signal of the user. The electrical energy may be an EEG of the user. Other electrical signals may also be observed. For example, the electrodes may be positioned to determine an intrinsic frequency of the user of the electrical activity of a user within an EEG band. Exemplary embodiments may obverse and/or measure the electrical signal of the user simultaneously with administering an energy source according to embodiments described herein. Exemplary embodiments may be configured to determine the effect of the administered energy on the measured signal in order to extract a user's measured signal without the presence of the administered energy. Exemplary embodiments may be configured to sequentially administer an energy source and before or after the administration of an energy source measure or receive a measurement from the user. Exemplary embodiments may use the measurement from the user to set a parameter of the administered energy after measurement.

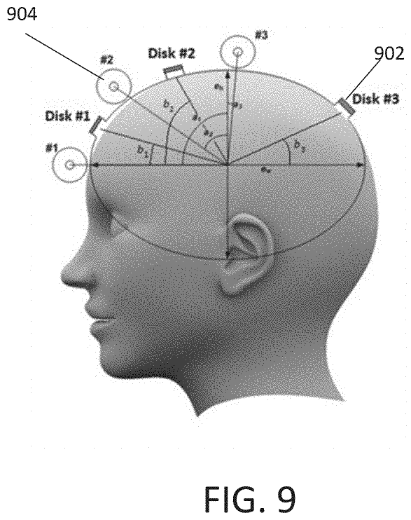

FIG. 9 is similar to FIG. 7A with the addition of the electrodes for measuring electrical activity in the brain. Although described as receiving electrical signals, the electrodes may also be used to administer electrical signals to the brain. Exemplary embodiments may include electrodes for receiving an electrical signal from the user's brain, while another set of electrodes may be used for generating an electrical signal to administer to the brain. Any combination of electrodes may be used and remain within the scope of the instant disclosure.

In an exemplary embodiment, the HMD includes a plurality of electrodes 902 and a plurality of magnetic energy sources 904 (whether permanent magnets and/or coils). The electrodes and magnetic energy sources may be positioned in alternating arrangement about the user's head. For example, the HMD may include three magnetic energy sources. The HMD may include an electrode positioned between each of the magnetic energy sources, and may include an electrode outside of the magnetic energy sources. As illustrated, a first electrode may be toward the forward part of the user's head, such as over the user's forehead. An electrode may be positioned toward the rear of the user's head. In an exemplary embodiment, the forward electrode and rearward electrode may be rotationally offset from the top of the user's head by approximately the same angle. An electrode may be positioned toward the top of a user's head. For example, a middle electrode may be positioned between the forward electrode and the rearward electrode. As illustrated, the middle electrode may be positioned toward the front of the head or toward the first electrode. The electrode may, for example, be angled forward toward the front of the head by approximately 25 degrees to about 30 degrees. As measured from the forward direction, or from the major axis of the ovoid, the first electrode may be at approximately 33 degrees, the second electrode at 63 degrees, and approximately at 147 degrees.

In an exemplary embodiment, the electrodes are used for sensing electrical signals from the user. In this case, the electrode in the forward position may be a sense electrode, the electrode toward the top of the head may be a reference electrode, and an electrode toward the back of the head may be a ground electrode.

Exemplary embodiments show and describe using electrodes to measure an electrical signal of the user. Other biometric signals and/or measurements of the user may be used in combination or alone. In an exemplary embodiment, a measurement system may be used to determine an intrinsic EEG frequency. This may be achieved even if a full EEG is not generated. In an exemplary embodiment the system may receive and/or record a peripheral nerve activity that is modulated by the intrinsic frequency. For example, the user may receive a flash of light at a certain frequency. The light frequency pulse may be altered and the system configured to receive an input to detect or to indicate when the use cannot distinguish the flashing and the light appears solid. This point may occur about twice the intrinsic frequency. Acoustic sounds may similarly or alternatively be used. For example, the system may play short beeps with the duration closer and closer together until the beeps become a constant tone to the perception of the user. The system may receive an input from the user to indicate when this occurs. The user interface, such as the user's mobile device may be used to administer the light or sound and/or receive the user input to indicate when the signal appears constant to the user. Other biological signals may also or alternatively be measured. For example a biometric, such as heart rate may be taken. Exemplary embodiments of additional measurement methods and devices may include infrared/fNIR technology.

In an exemplary embodiment, the interior surface of the HMD along all or portions of the surface may be in contact with a user's head. In an exemplary embodiment, the interior surface may therefore be at or less than 30° C. The configuration, material, temperature, and combinations thereof of the HMD is desirably comfortable or at least not uncomfortable to the user.

In an exemplary embodiment, the stimulation device according to embodiments described herein may include a vibration energy source. In an exemplary embodiment, the vibration energy source may be from a rotating and/or translating weighted material. The vibration may be generated by the permanent magnet and/or may be a separate component part. Attached hereto are exemplary embodiments of a vibration energy source and how it may be incorporated into a head mounted device. Any combination of these features may be included in the HMD described herein.

In an exemplary embodiment, the headset, controller, and/or user interface may include a feature to turn on and/or off and/or adjust the amplitude and/or adjust the frequency of the vibrational energy source. In an exemplary embodiment, a controlling device may be a knob.

In an exemplary embodiment, the stimulation device according to embodiments described herein may be visual stimulation. In an exemplary embodiment, the visual stimulation may be a colored light source. For example, the head mount display, controller, and/or user interface may include lights that can be observed during the treatment with the head mounted device.

In an exemplary embodiment, one or more LEDs may be positioned on the head mounted device. For example, along a front portion of the HMD, the HMD may include LEDs of at least three colors. In an exemplary embodiment the colors may be yellow, green, and blue, but other colors may also be used. The LEDs may be positioned such that a user can sense the color of the lights when wearing and operating the HMD, when the wearer is looking forward.

In an exemplary embodiment, the system may be configured to display a light to the user. The light may be an indicator as described herein. In an exemplary embodiment, the headset may be configured such that during treatment, the color of the light from the LEDs may change. For example with three colors, a first color (such as yellow) may be used for the first third of the treatment period, before the color transitions to a second color (such as green) for a second third of the treatment period, before finally transitioning to a third color (such as blue) for a final third of the treatment period. The light may be to assist in the treatment of the user. The light may provide a relaxing moot to conduct the treatment. The frequency of the light may be tuned with that of the treatment. In an exemplary embodiment, the user may select a color, such as through a control device on the HMD, controller, and/or user interface.

Exemplary embodiments of the system and methods described herein may be used as a general wellness device. For example, embodiments may include portable devices that may be used in an individual's home and administered by the user. It may therefore be used to maintain a general state of health. For example, systems and methods described herein may be used for relaxation, stress management, mental acuity, and sleep management. Exemplary embodiments described herein may use low energy fields induced in the body. Low energy fields are considered fields that are unable to cause neuronal depolarization. Accordingly, embodiments described herein are considered low risk to the user.

Exemplary embodiments of the systems and methods described here may include moving the activity of a user's brain to a different EEG band by stimulating the user's brain at a new frequency in that band. In an exemplary embodiment, the system and method may be used to gradually pull the intrinsic frequency of the user to a desired EEG band. For example, the system and/or methods may be used to improve sleep. The administered energy may be at an intrinsic frequency within the Theta Band of the EEG in order to pull the user's intrinsic frequency of the brain activity into the Theta Band to improve sleep.

The functional changes to the brain's neuronal activity as a result of stimulation at the brain's intrinsic frequency is that the brain becomes “tuned”, in that neuronal firings across the brain become more rhythmic, synchronous, and coherent. The increased brain synchronicity improves the person's focus, concentration, calmness, and may also improve the symptoms of a large number of mental disorders. When the brain is tuned, the EEG around the bandwidth of the intrinsic frequency distribution of a specified EEG band tends to narrow, resulting in an increased Q-factor. The Q-factor is defined as the ratio of the intrinsic frequency divided by the bandwidth of the frequency distribution about the intrinsic frequency. It is also possible to de-tune the brain of the person by imparting stimulation that is not at the brain's intrinsic frequency, resulting in a decreased Q-factor. For example, the stimulation frequency could be greater than 1 Hz away from the brain's intrinsic frequency. In another example, the stimulation frequency could shift randomly at periodic intervals to values that are not equal to the brain's intrinsic frequency.

If the frequency of stimulation is set to a value that is different from the intrinsic frequency, but within about ½ Hz from the intrinsic frequency, the stimulation energy tends to “pull” the intrinsic frequency toward the frequency of stimulation. Therefore, it is also possible to change the intrinsic frequency toward a pre-specified target value.

Since the energy of stimulation may be lowered when the frequency content of the energy is set to the person's intrinsic frequency, other lower energy modalities may be used, and still achieve an effect. For example, repetitive Transcranial Magnetic Stimulation (rTMS), transcranial Alternating Current Stimulation (tACS), ultrasound, sound, acoustic, light, radio frequency, and other energy forms may be used to provide very low energy to the brain. tACS uses electrodes on the scalp to deliver electrical pulses to the brain of the person through the skull, and if those electrical pulses are targeting the brain's intrinsic frequency of neuronal firing, then a change in brain functionality can be brought about, even though the stimulation is very low.

In one aspect are methods of treating a subject comprising: (a) adjusting the energy source at a frequency for influencing an intrinsic frequency, a measure of frequency selectivity and rhythmicity of a specified EEG band, of the person toward a pre-selected or target intrinsic frequency within a target band of the EEG; and (b) applying said energy to the head of the subject. The target band of the EEG may be in a different band the user is presently experience such that the system and method is configured to move the activity of a user into a different EEG band by stimulating the brain at a new frequency in the target band.

In one aspect are methods of treating a subject comprising: (a) adjusting the energy source at a frequency for influencing Q-factor, a measure of frequency selectivity and rhythmicity of a specified EEG band, of the person toward a pre-selected or target Q-factor of the band; and (b) applying said energy to the head of the subject.

In another aspect are methods of treating a subject comprising: (a) determining the Q-factor of the intrinsic frequency within the specified EEG band of the subject; (b) comparing the Q-factor of the intrinsic frequency from step (a) to an average Q-factor of the intrinsic frequency of a healthy population database. If the Q-factor of the intrinsic frequency from step (a) is higher than the average Q-factor of the intrinsic frequency of a healthy population database, tuning down the Q-factor of the intrinsic frequency of the subject by applying an energy with a plurality of frequencies or with a single pre-selected frequency close to a head of the subject; and if the Q-factor of the intrinsic frequency from step (a) is lower than the average Q-factor of the intrinsic frequency of the healthy population database, tuning up the Q-factor of the intrinsic frequency of the subject by applying energy to the head of the subject with a pre-selected frequency.

The energy may be generated by many different methods, and/or devices, and/or combinations thereof. For example, the energy may be ultrasonic, light, radio frequency, sound, acoustic, vibrational, electric, magnetic or combinations thereof. The energy may include a varying signal. Exemplary embodiments may include altering a frequency encountered by a user to be in relationship to, such as approximately equal to, a subharmonic of, or a harmonic of the administration frequency. For example, sounds the user may encounter, such as an alarm clock sound, cell phone or other telephone ring tone, timers, alerts, notifications, and other encountered sounds. The encountered sounds may be used to reinforce the effects of a protocol after a session has ended.

Exemplary embodiments may combine different forms of treatment. For example, magnetic stimulation generated through electrical current through coils may be combined with magnetic stimulation generated through rotating magnets. The combination of coils and rotating magnets may permit pulsed stimulation and sinusoidal stimulation simultaneously and/or sequentially. Exemplary embodiments may include administering energy stimulation at co-occurring, resonant, conflicting, or other parameter relationship. As an example, tACS may be combined with rTMS. If the administration of energy from the different energy sources is synchronous, then the effects to the user may be additive. Exemplary embodiments may include combining tDCS with rotating magnets. For example, anodal/excitatory tDCS may be used to reinforce neuronal firing with rotation of the permanent magnet, while cathodal tDCS may be used to provide more contrast between resting activity and flux generated by the magnetic energy stimulation.

Exemplary embodiments may combine administering energy according to embodiments described herein. In an exemplary embodiment, the administration of any combination of magnetic energy, electric energy, or vibrational energy may be combined with the administration of light or acoustic energy. The wavelengths of the light and/or tempo of the music may be selected to combine or match a frequency of another form of energy stimulation. Exemplary embodiments may use harmonics and/or subharmonics to create a matching frequency. In an exemplary embodiment, the administration of an energy stimulus is combined with music. The typo of the music may be synchronous with the frequency of the administered energy source (such as magnetic or electric frequency). The tempo of the music may be synchronous with a target administration frequency, such as a target intrinsic frequency of the user in an EEG band. Exemplary embodiments may provide a music selection to play automatically with a treatment session. Exemplary embodiments, may permit the user to select music options to play during a treatment session. The system may modify the music such that it may run faster or slower so the rhythm of the music matches a harmonic of the administration frequency of another energy source and/or to match a frequency or subharmonic or harmonic of the target treatment frequency. Music or other sounds may by modulated to create a warble at the target treatment frequency. Other sounds may be used instead of a music. A sound may be modulated or played according or in relation to the target administration frequency. For example, rain drops, white noise, or other repetitive noise may be used at a specified frequency, where the specified frequency has a relationship to the target administration frequency. The phase relationship between the administration of an energy source at a target frequency may be set against the modulation of the audio signal (the warble) to achieve different effects. Light may be modulated in simular fashions. The light may be modulated to change colors and/or change amplitude at a desired frequency. The desired frequency may be at a target administration frequency and/or at a harmonic or subharmonic thereto. In an exemplary embodiment, the light may be pulsed. As described herein, the target administration frequency, target frequency, specified frequency or other frequency described herein, may be different frequency used in administering an energy source to a user to achieve a desired effect within the user. These frequencies may be in relationship to an EEG of a user. For example, the frequencies may be in relationship, such as a harmonic or sub-harmonic of or approximately equal to or in a desired direction from the measured frequency of a user of an intrinsic frequency of a user.

The administration of one or more sources of energy stimulation may also be used in combination with other activities and/or administration of other treatments or inputs to the user. The monitoring and/or stimulation may be used to improve performance by the user at the activity. The monitoring and/or stimulation may be able to improve the user's retention of the activity. The monitoring and/or stimulation may be used to improve the effect on the user of the other activity or input.

In an exemplary embodiment, the administration of energy stimulation may be used in combination with activities to assist in the activity being performed. For example, the user may undergo treatments according to embodiments described herein while performing a cognitive activity. Exemplary cognitive activities may include any learning and/or memory activity. Exemplary embodiments of the administration of energy described herein may be used to improve concentration, wakefulness, attention, retention, and combinations thereof. For example, exemplary embodiments used herein may be administered while a user is receiving information about a new language. The user may improve cognitive function to improve retention of the new language. Other activities that may be combined with administration of energy as described herein may include activities of fine motor skills or tasks. High frequency administration of energy sources may be used in one or more embodiments.

Exemplary embodiments may include other cognitive activities requiring concentration. For example, the monitoring by EEG or electric signal activity of the user from the brain may be used to assess a focus or concentration of a user. The system may analyse the received information and assess whether the user is losing focus, or falling asleep, or changing into another cognitive state. The system may thereafter request user input and/or may administer a protocol to improve, restore, or maintain the cognitive state. For example, exemplary embodiments used herein may monitor the cognitive state of a user that is intended to keep watch. This may include drivers, soldiers or security guards, pilots, etc. The system may detect when the user's attention drops below a desired level and/or moves away from a set condition and/or enters a predefined condition. Any of the conditions, levels or other parameter may be used to indicate that a focus of the user is altered and/or diminished, such as if the user is falling asleep. The system may, for example, monitor a theta band of an EEG to determine if a user is falling asleep. Upon detection of the parameter in comparison to a predefined condition, the system may administer a protocol. The protocol may be an administration of one or more energy sources at a desired target frequency according to embodiments described herein. For example, the administration of the target frequency may be to move the user away from the theta band and into the alpha band to improve cognitive focus and awareness. Exemplary embodiments may therefore be integrated into hats, helmets, or other hardware used in the situation of the user, such as in hard hats, pilot helmets, etc.

Exemplary embodiments may also incorporate the monitoring of a user according to embodiments described herein to determine a condition of the user and/or monitor the continued condition of the user for a change. The system may be configured to provide the user an alert if the condition changes. The system may be configured to provide an alert to one or more system components connected to the system, such as other electronic devices and/or computers described herein. The system may therefore provide notice to a system administrator, manager, or other person that may need to know the condition of the user. Exemplary embodiments may include monitoring of the user. Once a condition change is detected, the user may be instructed to receive treatment. The user may thereafter go to a treatment location and administer a treatment according to embodiments described herein.

Exemplary embodiments are not limited to cognitive activities, but may also include physical activities. Exemplary embodiments may use embodiments described herein as a user performs a physical activities, such as participating in a work out or undergoing physical therapy. Exemplary embodiments may be combined with a rhythmic exercise or physical activity. In an exemplary embodiment, the pace of the activity may be at, a harmonic of, or subharmonic of the target frequency. The target frequency may be an intrinsic frequency of the user's EEG in a desired band. The target frequency may be a desired intrinsic frequency of an EEG band the user is trying to attain. Performance of a rhythmic exercise may be used to improve the results of the administration of the energy according to embodiments described herein. The rhythmic exercise may be performed synchronously with the administration of an energy source as described herein.

In an exemplary embodiment, a biological attribute or function of the user may be adjusted to correspond to a frequency, harmonic of, or subharmonic of the target or administered frequency. An exercise regimen may be used to bring the user's heartrate to a desired rate. The intensity of an exercise or physical activity may be used to bring the heart rate to a point where it is a harmonic of the administration frequency of the energy source. For example, if the administration frequency is 10 Hz, a user may use a stationary bicycle or pedal system which adjusts the resistance to bring the heart rate to 2 Hz (120 bpm), which is the 5^{th} subharmonic of the administration frequency.

Exemplary embodiments may be administered and/or used with the administration of other treatment protocols. For example, a user may be taking antidepressants. Due to the neurogenesis properties of the SNRIs, neurons may be created in the brain. However, the formed neurons may be nonfunctional due to a lack of connectivity with neighboring brain tissue. Exemplary embodiments of the methods described herein may be to administer an energy source to the brain of the user at an administration frequency after the patient has taken an antidepressant. The synchronous treatment may assist in creating and strengthening neuron connectivity within the brain and improve the results of the antidepressants. Exemplary embodiments may include taking supplements, eating food, etc. to achieve improved results. Exemplary embodiments described herein may be incorporated or used with any brain impacting medication and/or treatment. For example, medications that impact serotonin, dopamine, glutamate, etc. Exemplary embodiments of energy stimulation may be combined with these medications to amplify or interact with the method of administration of the medication. For example, the administration of an energy stimulation with a dopamine reuptake inhibitor (DRI) may result in a lower dosage of the DRI to have the same efficacy, due to the combined effect of more extracellular concentration of dopamine (due to DRI) and an increase in dopamine production from the energy stimulation.

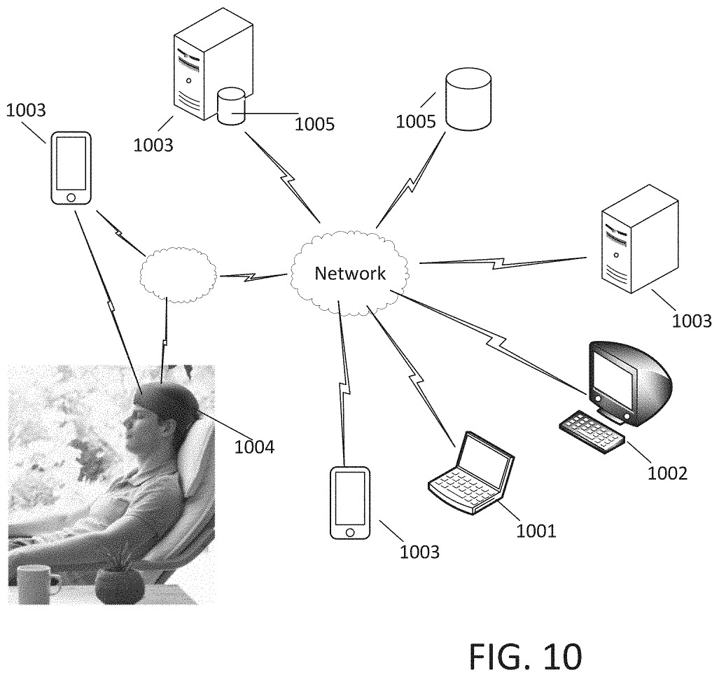

FIG. 10 illustrates exemplary system that can include energy stimulation to a user as described herein. Exemplary embodiments of the system described herein may include a computer, computers, electronic device, or electronic devices. As used herein, the term computer(s) and/or electronic device(s) are intended to be broadly interpreted to include a variety of systems and devices including personal computers 1002, laptop computers 1002, mainframe computers, servers 1003, set top boxes, digital versatile disc (DVD) players, mobile phone 1003, tablet, smart watch, smart displays, televisions, and the like. A computer can include, for example, processors, memory components for storing data (e.g., read only memory (ROM) and/or random access memory (RAM), other storage devices, various input/output communication devices and/or modules for network interface capabilities, etc. For example, the system may include a processing unit including a memory, a processor, an analog-to-digital converter (A/D), a plurality of software routines that may be stored as non-transitory, machine readable instruction on the memory and executed by the processor to perform the processes described herein. The processing unit may be based on a variety of commercially available platforms such as a personal computer, a workstation a laptop, a tablet, a mobile electronic device, or may be based on a custom platform that uses application-specific integrated circuits (ASICs) and other custom circuitry to carry out the processes described herein. Additionally, the processing unit may be coupled to one or more input/output (I/O) devices that enable a user to interface to the system. By way of example only, the processing unit may receive user inputs via a keyboard, touchscreen, mouse, scanner, button, or any other data input device and may provide graphical displays to the user via a display unit, which may be, for example, a conventional video monitor. The system may also include one or more large area networks, and/or local networks for communicating data from one or more different components of the system. The one or more electronic devices may therefore input a user interface for displaying information to a user and/or one or more input devices for receiving information from a user. The system may receive and/or display the information after communication to or from a remote server 1003 or database 1005.

As illustrated, the system may include software stored in memory 1005 and accessed by a computer 1003, such as a remote server. The head mounted device 1004 may communicate either through an electric device, such as a mobile phone 1003, or through a network with the remote server to receive treatment protocol instructions for the user. The system may therefore be configured to retrieve user information, such as an EEG or electrical signal from the electrodes and analyse the information. The information may be analysed locally, such as a computer in communication with the headset and/or may be communicated to the remote server for the remote server to process the data and provide treatment protocols back to the HMD.

Exemplary embodiments described herein include determining an intrinsic frequency of a user's EEG, and/or administering an energy source based on the intrinsic frequency (whether measures from the user or a target intrinsic frequency). In an exemplary embodiment, the intrinsic frequency may be measured and determined by determining a maximum energy based on an FFT (Fast Fourier Transform). Other methods may be used to determine the intrinsic frequency. For example, the system may use curve fitting of an electric signal, wavelet transforms, or other approximation or wave analysis from the user's brain and determine an intrinsic frequency. Exemplary embodiments may include determining a higher frequency local maxima. Exemplary embodiments may determine an intrinsic frequency by looking for higher frequency local maxima in a detected or measured electrical signal from the user's brain. In an exemplary embodiment, an evoked activity with stimulation searching may be used. For example, a wideband stimulation may be provided. The bands of stimulation may be provided by selecting a given frequency and hopping the frequency about the band, or setting a predetermined frequency band, such as 8-9, 8-10, 9.9.5 Hz) and then observing for elicitation of oscillations not at dominant rhythm. By searching at higher harmonics via set parameters (such as 1 Hz, 0.5 Hz, 1.5 Hz, or 2 Hz) or those derived from biometrics (such as heart rate, or as related to respiration) or those derived from EEG (such as identification of largest 2-3 components in the alpha EEG band), and determining if those at higher frequencies are viable targets for stimulation. Different weights based on EEG location (a frontal rhythm at a voltage of n is less likely to be chosen than a faster occipital rhythm at a voltage of <n) may be used to determine a likely target frequency.

Exemplary embodiments of the system and methods described herein may include using wavelet analysis to determine stimulation frequency. Exemplary embodiments may use wavelet analysis such as the continuous wavelet transform (CWT) to measure EEG and/or other biometric signals in order to contribute to a treatment protocol. Exemplary embodiments of using wavelets may also be used in closed-loop feedback methods to inform and/or make decisions within the system operation and/or monitoring. In an exemplary embodiment, wavelets may be used as a method by which extraneous noise/artefacts in signals may be ignored. Mother wavelets of any shape may be used to measure all or any portion of the activity described herein.

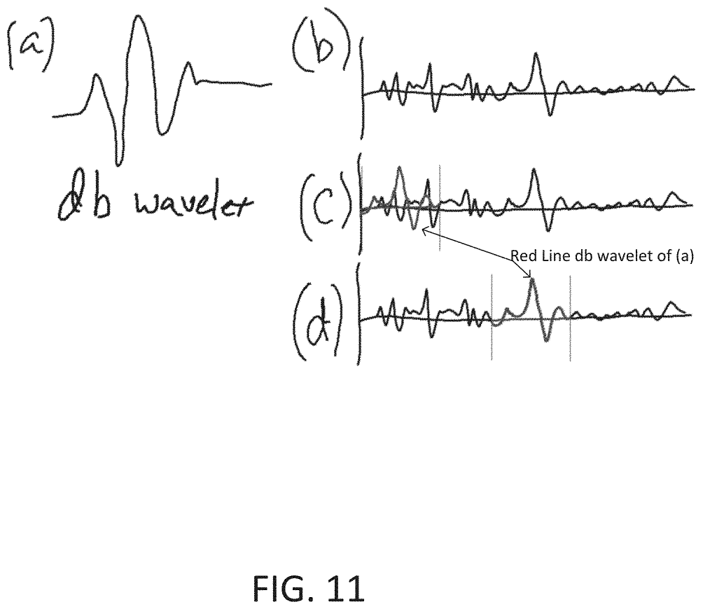

FIG. 11 illustrates an exemplary wavelet and application of the wavelet to an EEG wave form according to embodiments described herein.

EEG oscillations are dynamic in presentation and vary in frequency, amplitude and occurrence, with waveform shape and overlap common in a recording. FFT analysis and curve-fitting approaches may estimate the presence and density of specific re-occurring EEG waveforms (to be known as a ‘wavelet’ or ‘wavelets’) in comparison to other wavelets of varying shape and frequency. However they do not specifically search for or precisely identify wavelets in time.

The wavelet transform (WT) is a method by which a waveform is defined, in both shape, magnitude and frequency, such that it fits and overlaps with wavelets of varying frequency, shape, and magnitude and can thus be applied to time-series of biometric data for specific identification of wavelets that fit to the specification of the WT. The WT may be applied for removal of noise in the signal, and/or for feature identification and extraction. The WT can define multiple base waveforms, known as ‘mother wavelets,’ including but not limited to Morlet, Daubechies, Biorthogonal, Orthogonal Cubic Spline, Coiflets, Complex Gaussian, Mexican Hat, and Haar wavelets, in addition to other wavelets—including those in discrete and continuous wavelet classes. Wavelets in a WT may be applied with different filtering settings (coefficients) such that the mother wavelets can be adjusted in size to identify any possible frequency of wavelet available in the signal, at every time point in the signal.

FIG. 11 illustrates an exemplary application. FIG. 11 (a) illustrates an exemplary Wavelet Transform using a Daubechies wavelet matching the stimulation frequency. For example, the stimulation may be provided at 10.6 Hz. In the exemplary application, an EEG may be scanned following, during, or between stimulation provided at 10.6 Hz with a WT using a Daubechies wavelet matching the stimulation frequency. The exemplary EEG of a patient is seen in FIG. 11(b), which is reproduced in FIGS. 11(c)-11(d). As seen in FIGS. 11(c)-11(d), the Wavelet Transform (red) is compared against EEG signal (black). Increases in the wavelet of interest at 10.6 Hz as measured by the WT may influence stimulation parameters, including reducing the duration of total stimulation, or influencing when stimulation is to be next administered, or other characteristics of stimulation. Referring more specifically to FIGS. 11(a)-(d), (a) defines a Daubechies wavelet (db) with some coefficient values such that a waveform of interest may be searched for in an EEG signal. An EEG signal (b) is scanned with the db WT (c) until a corresponding wavelet is noted in the EEG (d) at which time stimulation may be delivered or treatment parameters may be adjusted in some fashion.

Exemplary systems and methods may include using wavelet transforms to measure responses to therapy and adjustment to treatment parameters due to changes in occurrence of one or more type of wavelets (such as slow frequency wavelets) in relation to other wavelets (such as alpha wavelets) in specific time sequences using any combination of mother wavelet and type of WT. An example observation may be in a change in the series of slow wavelets followed by faster wavelets, or a difference in the overlapping of wavelets of different frequencies.

Exemplary systems and methods may include using WT to measure onset of specific wavelets following or prior to stimulation as a way to modify stimulation. An example includes recording EEG until a specific wavelet occurs and is identified by the WT, and then commencing stimulation immediately during or following the noted wavelet. Exemplary embodiments including commencing treatment after a series of the same wavelet occurs, such that stimulation only occurs after 2 or 3 wavelets of the same shape and frequency occur in the EEG or biometric time series.

Exemplary systems and methods may include using WT to specify components of stimulation (such as, for example, time, frequency, or a combination thereof), such that the EEG or biometric signal is monitored following a train of stimulation. Based on the presence of specific wavelets following stimulation (one or multiple), the same stimulation is repeated, or some component of the stimulation is altered and then additional WT is applied following the altered stimulation.

Exemplary embodiments of the systems and methods described herein using FT measures and/or curve fitting may also take advantage of and/or use wavelet transforms.

Exemplary embodiments of the systems and methods described herein may use machine learning and/or neural network algorithms and techniques. Machine learning (ML) involves the training of computer algorithms with the input of massive amounts of data. Derivations of ML include neural networks (NN), wherein an algorithm is trained to perform some task given a large training set. ML and NN may be used to train a predictive algorithm such that upon input of new data into the trained algorithm, a conclusion may be determined, within a percentage of certainty or confidence interval. Training data may include EEG and/or biometric sensory data from all or some of the EEGs collected on the user or on all users. The training and update of the NN algorithm may be updated at any time, with any addition of data. NN may use any measurement of supplied data, including FFT, curve fitting, WT and other measurements of data provided to the NN, incorporating measurements as part of the algorithm training process. New analysis techniques of already provided data may be supplied to the NN at any time to further augment the NN algorithm(s). The trained task may be, for example, the identification of an EEG signature that is sensitive to specific stimulation parameters, whether those parameters be duration of stimulation, frequency of stimulation, total duration of a stimulation session, other components of stimulation, and any combination thereof.

In one example, a subject's EEG and biometric data is recorded by the system and a trained NN indicates that the subject will be more responsive to a longer period of stimulation, only with the front-most magnet involved in the session: and stimulation parameters are adjusted accordingly. In another example a subject receiving stimulation for 20 minutes of a 30 minute stimulation session is monitored by a NN, which indicates (as a result of biometric data gathered during the 20 minute session) that additional stimulation is most likely not additive, and the stimulation session is halted immediately.

NN may be used to influence stimulation parameters prior to, during, or in-between stimulation. NN may be used to shift stimulation parameters not only for magnetic field stimulation, but also for the integration of magnetic stimulation and other stimulation types, including auditory, and visual stimulation. For example, during the first session of stimulation, stimulation is delivered at varying frequencies from varying magnets while a NN monitors EEG and biometric data and once enough data is gathered, specific stimulation settings are chosen or influenced by the NN based on the biometric response to the varying stimulation.

In an exemplary embodiment, NN may be used to provide feedback to the user about the expected durations of treatment, number of treatment sessions, or other information about treatment.

In an exemplary embodiment, NN may be used to monitor a subject with the headset and provide stimulation when the NN predicts a critical value may, with high confidence, develop. For example, the user may be wearing the headset while working on heavy machinery and the NN monitoring biometric data indicates that the subject is falling asleep, at which time stimulation may be delivered.

Exemplary systems and methods described herein include a modular and/or mobile device that may be adapted for use in many different environments. For example, embodiments described herein may be included in a kiosk or sit down administration station that may be used at a pharmacy or walk up location. The system may therefore permit easy cleaning and/or use of disposable parts that may be in contact with different users. For example, the outer layer of the HMD may be disposable and include a wearable and adjustable band of sturdy paper type material intended for retaining the energy sources in a desired location on a user's head for a single use application. The inner portion of the HMD may be easily wiped clean with a disinfectant or put in a disinfecting compartment of the kiosk between uses. The disinfecting compartment may administer alcohol, ultraviolet rays, or other antimicrobial, antiseptic, antiviral cleaning agent or mechanism. Similarly, exemplary embodiments may be integrated into seats, such as in headrests. Exemplary embodiments may be used as an upgrade feature for the seat. For example, for use with recliners or home use chairs, a user may purchase an add-on, including a pluggable extension or HMD according to embodiments described herein for use with the purchased chair. Airlines (or other transit form, such as a bus or train) may similarly permit the purchase of a treatment session for use during a flight or other travel period. The HMD may therefore be used as a stand-alone device or may be integrated into a seat of the transportation vehicle.

Exemplary embodiments of the system described herein can be based in software and/or hardware. While some specific embodiments of the invention have been shown the invention is not to be limited to these embodiments. For example, most functions performed by electronic hardware components may be duplicated by software emulation. Thus, a software program written to accomplish those same functions may emulate the functionality of the hardware components in input-output circuitry. The invention is to be understood as not limited by the specific embodiments described herein, but only by scope of the appended claims.

Although embodiments of this invention have been fully described with reference to the accompanying drawings, it is to be noted that various changes and modifications will become apparent to those skilled in the art. Such changes and modifications are to be understood as being included within the scope of embodiments of this invention as defined by the appended claims. Specifically, exemplary components are described herein. Any combination of these components may be used in any combination. For example, any component, feature, step or part may be integrated, separated, sub-divided, removed, duplicated, added, or used in any combination and remain within the scope of the present disclosure. Embodiments are exemplary only, and provide an illustrative combination of features, but are not limited thereto.

When used in this specification and claims, the terms “comprises” and “comprising” and variations thereof mean that the specified features, steps or integers are included. The terms are not to be interpreted to exclude the presence of other features, steps or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

What is claimed is:

1. A system for administering stimulation energy to a user, comprising:
a head mounted device configured to be positioned near the user's head;
at least one stimulation source configured to provide energy to the user's head during use.

2. The system of claim 1, wherein the head mounted device is configured to fit on a portion of the user's head.

3. The system of claim 2, wherein the at least one stimulation source comprises a first magnet, a second magnet, and a third magnet, wherein each of the first magnet, the second magnet, and the third magnet are cylindrical magnets configured to rotate about an axis of symmetry of each of the first magnet, second magnet, and third magnet.

4. The system of claim 3, wherein the first magnet is positioned at a front of the head mounted device, the third magnet is positioned at a top of the head mounted device, and the second magnet is positioned between the first magnet and the third magnet.

5. The system of claim 4, further comprising a controller in communication with the head mounted device for controller a speed of rotations of the first magnet, the second magnet, and the third magnet.

6. The system of claim 5, wherein the head mounted device comprises a band positioned around a lower terminal end of the head mounted device for securing the head mounted device to the user's head and is configured to maintain a relative position of the head mounted device relative to the user's head during use.

7. The system of claim 6, wherein the band is flexible to conform to the user's head during use.

8. The system of claim 7, wherein the head mounted device comprises a rigid housing for enclosing the at least one stimulation source.

9. The system of claim 8, further comprises one or more electrodes for receiving an electrical signal from a user's brain during use.

10. The system of claim 9, wherein the head mounted device comprises one or more indentions, wherein each of the one or more indentions are configured to receive one of the one or more electrodes.

11. The system of claim 10, wherein a first electrode is positioned between the first magnet and the second magnet, a second and third electrode are positioned on an opposite side of the third magnet away from the first magnet.

12. A method of administering stimulation to a user, comprising:
providing a head mounted device near a patient's head, the head mounted device having an energy source;
providing energy to the patient's head with the energy source.
